(19) United States
(12) Patent Application Publication
(10) Pub. No.: US 2006/0222693 A1
(43) Pub. Date: Oct. 5, 2006

(54) Transdermal patch bearing a decorative image

(76) Inventors: Kandy Michelle Silguero, Harlingen, TX (US); Rose Silguero, Robstown, TX (US); Brenda Ramon, Robstown, TX (US)

Correspondence Address: LAW OFFICES OF ERIC KARICH, 2807 ST. MARK DR., MANSFIELD, TX, 76063, US

(21) Appl. No.: 11/095,145

(22) Filed: Mar. 30, 2005

(51) Int. Cl. A61K 9/70

(52) U.S. Cl. 424/449

(57) ABSTRACT

A transdermal patch is disclosed for administering a drug to a human user. The transdermal patch includes a pigment layer having an image formed therein, a layer including the drug, and an adhesive layer for adhering the transdermal patch to the skin.

### BACKGROUND OF THE INVENTION

1. Field of the Invention

This invention relates generally to drug administering apparatus, and more particularly to a drug administering apparatus that resembles a decorative feature or tattoo.

2. Description of Related Art

In general, a transdermal patch is a medicated adhesive pad placed on the skin to deliver a medication through the skin. Transdermal patches are commonly used to administer certain types of drugs to human users. As used herein, the term “drug” is defined as a substance that is either: (i) used in the diagnosis, treatment, or prevention of an unwanted condition or disease, (ii) recognized or defined by Title 12, Chapter 9 of the U.S. Code (the Federal Food, Drug, and Cosmetic Act) to be a drug, or (iii) affects the central nervous system causing a change in behavior.

In general, a tattoo is a graphic image made on the skin by a process of pricking and ingraining an indelible pigment, or by raising scars. During the past decade tattoos have become more and more common in American society, as a means of creative self-expression.

### SUMMARY OF THE INVENTION

A transdermal patch is disclosed for administering a drug to a human user. The transdermal patch includes a pigment layer having an image formed therein, a layer including the drug, and an adhesive layer for adhering the transdermal patch to the skin.

Other features and advantages of the present invention will become apparent from the following more detailed description, taken in conjunction with the accompanying drawings, which illustrate, by way of example, the principles of the invention.

### BRIEF DESCRIPTION OF THE DRAWING

The accompanying drawings illustrate the present invention. In such drawings:

FIG. 1 is a perspective view of a female user having one embodiment of a transdermal patch attached to skin of her abdomen;

FIG. 2 is a top plan view of the transdermal patch of FIG. 1;

FIG. 3 is a cross sectional view of one embodiment of the transdermal patch of FIGS. 1 and 2; and

FIG. 4 is a cross sectional view of another embodiment of the transdermal patch of FIGS. 1 and 2.

### DETAILED DESCRIPTION OF THE INVENTION

FIG. 1 is a perspective view of a female user 10 having one embodiment of a transdermal patch 12 attached to skin of her hip. In general, the transdermal patch 12 is used to administer one or more drugs to the user 10 via the skin. The transdermal patch 12 preferably releases the one or more drugs into the skin at a controlled rate, and preferably over a significant period of time. The one or more drugs may include, for example, nicotine (to help the user 10 stop smoking), diet drugs (to help the user 10 lose weight), or one or more hormones that prevent the female user 10 from ovulating (to prevent the female user 10 from becoming pregnant).

In the embodiment of FIG. 1, the transdermal patch 12 has an adhesive layer for adhering the transdermal patch 12 to the skin of the user 10. It is noted that the transdermal patch 12 can be adhered to skin on many other regions of the body of the user 10, including but not limited to an arm, a leg, the chest, or the back of the user 10. It is also noted that the transdermal patch 12 can be used to administer one or more drugs to male users as well as female users.

As described in more detail below, the transdermal patch 12 includes a layer bearing the one or more drugs, and a pigment layer having an image formed therein. The image is preferably a graphic image substantially made by applying lines and strokes to a two-dimensional surface, and preferably resembles a tattoo. In FIG. 1, the image resembles a tattoo of a butterfly. The image advantageously transforms the transdermal patch 12 from an otherwise unattractive appliance into a decoration and/or a form of body art.

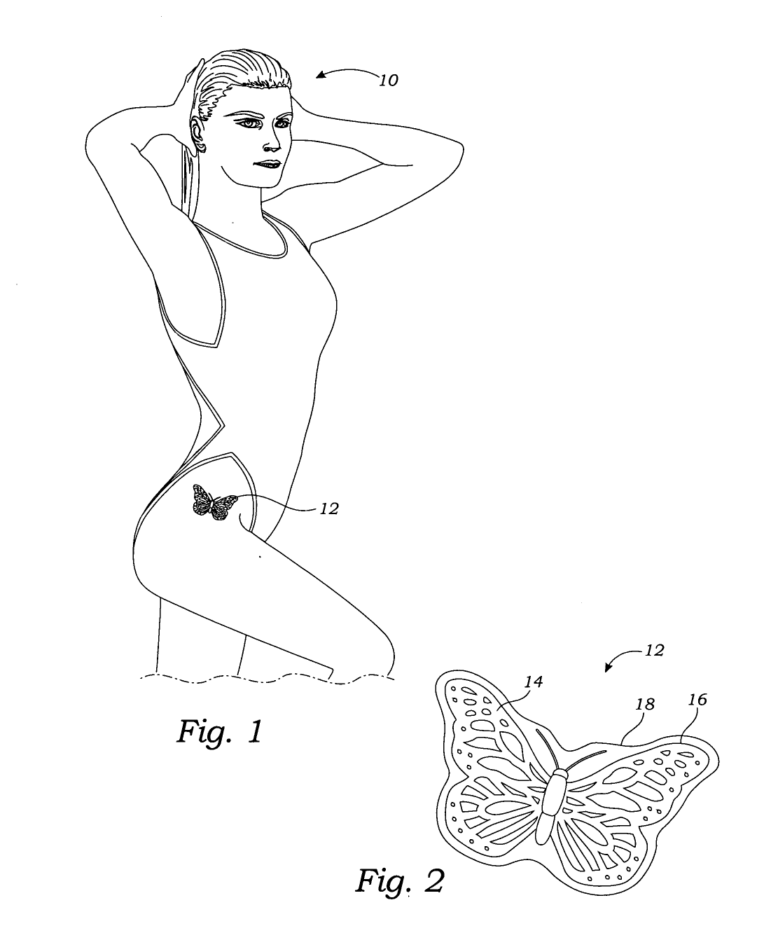

FIG. 2 is a top plan view of the transdermal patch 12 of FIG. 1. In FIG. 2 the image resembling a tattoo of a butterfly and formed in the pigment layer is labeled 14. The pigment layer is described in more detail below. The image 14 has an outer edge 16. In general, the transdermal patch 12 has a perimeter 18 that substantially conforms to the outer edge 16 of the image 14. The perimeter 18 of the transdermal patch 12 is also shaped to add structural support to the transdermal patch 12 where necessary. For example, In FIG. 2 the perimeter 18 of the transdermal patch 12 is farther away from the outer edge 16 of the image 14 in a region including antennae of the butterfly depicted in the image 14. This shaping of the perimeter 18 prevents the protruding and delicate antennae features from being separated from the transdermal patch 12 when a release liner layer is removed from the transdermal patch 12 prior to application of the transdermal patch 12 to the skin as described below, and when the transdermal patch 12 is removed from the skin after use.

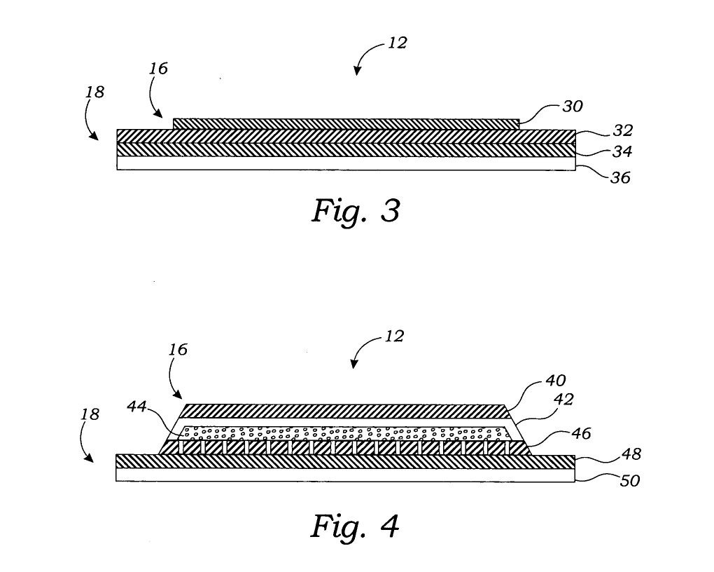

FIG. 3 is a cross sectional view of one embodiment of the transdermal patch 12 of FIGS. 1 and 2. In the embodiment of FIG. 3, the adhesive layer is labeled 34, and is also the layer that bears the one or more drugs. That is, the one or more drugs are dispersed in the adhesive layer 34, and adhesive layer 34 also serves to adhere the transdermal patch 12 to the skin of the user 10 (see FIG. 1). A release liner layer 36, in contact with a bottom surface of the adhesive layer 34 in FIG. 3, serves to protect the adhesive layer 34 prior to use of the transdermal patch 12. The release liner layer 36 is removed from the transdermal patch 12 prior to applying the transdermal patch 12 to the skin of the user 10.

In the embodiment of FIG. 3, the transdermal patch 12 also includes a backing layer 32 in contact with a top surface of the adhesive layer 34. The backing layer 32 prevents escape of the one or more drugs from the transdermal patch 12 (other than into the skin of the user 10 of FIG. 1), and also protects the transdermal patch 12 from degrading factors in the environment. For example, the backing layer 32 preferably prevents evaporation and/or other loss of the one or more drugs, and preferably protects the transdermal patch 12 from physical abrasion and/or from moisture in the environment.

In the embodiment of FIG. 3, the pigment layer having the image 14 formed therein (see FIGS. 1 and 2) is labeled 30, and forms an outermost layer of the transdermal patch 12 such that the image 14 is highly visible. In FIG. 3, the pigment layer 30 is in contact with a top surface of the backing layer 32. In general, the pigment layer 30 includes one or more pigments used to form the image 14. The one or more pigments are preferably non-toxic pigments.

The backing layer 32 and the adhesive layer 34 are preferably formed from materials that are substantially transparent or translucent and highly flexible in all directions. Suitable materials for the pigment layer 30, the backing layer 32, the adhesive layer 34, and the release liner layer 36 are known and commercially available.

In the embodiment of FIG. 3, an outer edge of the pigment layer 30 defines the outer edge 16 of the image 14 (see FIG. 2), and outer edges of the backing layer 32 and the adhesive layer 34 define the perimeter 18 of the transdermal patch 12. As described above, the perimeter 18 of the transdermal patch 12 is shaped to cooperate with the outer edge 16 of the image 14.

FIG. 4 is a cross sectional view of another embodiment of the transdermal patch 12 of FIGS. 1 and 2. In the embodiment of FIG. 4, the adhesive layer is labeled 48, and serves to adhere the transdermal patch 12 to the skin of the user 10 (see FIG. 1). A release liner layer 50, in contact with a bottom surface of the adhesive layer 48, serves to protect the adhesive layer 48 prior to use of the transdermal patch 12. The release liner layer 50 is removed from the transdermal patch 12 prior to applying the transdermal patch 12 to the skin of the user 10.

In the embodiment of FIG. 4, the transdermal patch 12 also includes a drug reservoir layer 44 containing the one or more drugs, and a membrane layer 46. A bottom surface of the membrane layer 46 is in contact with a top surface of the adhesive layer 48, and a bottom surface of the drug reservoir layer 44 is in contact with a top surface of the membrane layer 46. In general, the membrane layer 46 serves to control escape of the one or more drugs from the transdermal patch 12 into the skin of the user 10 (see FIG. 1). More specifically, the membrane layer 46 serves to control rates at which the one or more drugs enter the skin of the user 10.

In the embodiment of FIG. 4, the transdermal patch 12 also includes a backing layer 42 that surrounds the drug reservoir layer 44 on all sides except a bottom side. The backing layer 42 prevents escape of the one or more drugs from the transdermal patch 12 (other than into the skin of the user 10 of FIG. 1), and also protects the transdermal patch 12 from degrading factors in the environment. For example, the backing layer 42 preferably prevents evaporation and/or other loss of the one or more drugs, and preferably protects the transdermal patch 12 from physical abrasion and/or from moisture in the environment.

In the embodiment of FIG. 4, the pigment layer having the image 14 formed therein (see FIGS. 1 and 2) is labeled 40, and forms an outermost layer of the transdermal patch 12 such that the image 14 is highly visible. In FIG. 4, the pigment layer 40 is in contact with a top surface of the backing layer 42. In general, the pigment layer 40 includes one or more pigments used to form the image 14. The one or more pigments are preferably non-toxic pigments.

The backing layer 42, the drug reservoir layer 44, the membrane layer 46, and the adhesive layer 48 are preferably formed from materials that are substantially transparent or translucent and highly flexible in all directions. Suitable materials for the pigment layer 40, backing layer 42, the drug reservoir layer 44, the membrane layer 46, and the adhesive layer 48 are known and commercially available.

In the embodiment of FIG. 4, an outer edge of the pigment layer 40 defines the outer edge 16 of the image 14 (see FIG. 2), and an outer edge of the adhesive layer 48 defines the perimeter 18 of the transdermal patch 12. As described above, the perimeter 18 of the transdermal patch 12 is shaped to cooperate with the outer edge 16 of the image 14.

It is noted that other embodiments of the transdermal patch 12 of FIGS. 1 and 2 are possible and contemplated. Alternative shapes, printed images, and other decorative features, are all expressly considered within the scope of the present invention, and should be construed to be within the scope of the invention as claimed.

While the invention has been described with reference to at least one preferred embodiment, it is to be clearly understood by those skilled in the art that the invention is not limited thereto. Rather, the scope of the invention is to be interpreted only in conjunction with the appended claims.

What is claimed is:

1. A transdermal patch for administering a drug to a human user, the transdermal patch comprising:
a pigment layer having an image formed therein;
a layer comprising the drug; and
an adhesive layer for adhering the transdermal patch to the skin.

2. The transdermal patch as recited in claim 1, wherein the transdermal patch has a perimeter that substantially conforms to an outer edge of the image.

3. The transdermal patch as recited in claim 2, wherein an outer edge of the pigment layer defines the outer edge of the image, and wherein an outer edge of the adhesive layer defines the perimeter of the transdermal patch.

4. The transdermal patch as recited in claim 1, wherein the image resembles a tattoo.

5. The transdermal patch as recited in claim 1, wherein the adhesive layer comprises the drug.

6. The transdermal patch as recited in claim 1, wherein the transdermal patch is adapted to release drug into the skin at a controlled rate.

7. The transdermal patch as recited in claim 1, further comprising a backing layer adapted to prevent escape of the drug from the transdermal patch other than into the skin, wherein the backing layer is positioned between the pigment layer and the layer comprising the drug.

8. The transdermal patch as recited in claim 1, further comprising a release liner layer in contact with the adhesive layer and adapted to protect the adhesive layer prior to use of the transdermal patch.

9. The transdermal patch as recited in claim 1, wherein the layer comprising the drug comprises a drug reservoir layer.

10. The transdermal patch as recited in claim 9, further comprising a backing layer adapted to prevent escape of the drug from the transdermal patch other than into the skin, wherein the backing layer is positioned between the pigment layer and the drug reservoir layer.

11. The transdermal patch as recited in claim 10, wherein the backing layer surrounds the drug reservoir layer on all sides except a bottom side.

12. The transdermal patch as recited in claim 11, further comprising a membrane layer adapted to control escape of the drug from the transdermal patch into the skin, wherein the membrane layer is positioned between the drug reservoir layer and the adhesive layer.

13. A transdermal patch for administering a drug to a human user, the transdermal patch comprising:
a pigment layer having an image formed therein;
a layer comprising the drug;
an adhesive layer for adhering the transdermal patch to the skin;
wherein an outer edge of the pigment layer defines an outer edge of the image;
wherein an outer edge of the adhesive layer defines a perimeter of the transdermal patch; and
wherein the perimeter of the transdermal patch substantially conforms to the outer edge of the image.

14. The transdermal patch as recited in claim 13, wherein the image resembles a tattoo.

15. The transdermal patch as recited in claim 13, wherein the adhesive layer comprises the drug.

16. The transdermal patch as recited in claim 13, wherein the transdermal patch is adapted to release drug into the skin at a controlled rate.

17. The transdermal patch as recited in claim 13, further comprising a backing layer adapted to prevent escape of the drug from the transdermal patch other than into the skin, wherein the backing layer is positioned between the pigment layer and the layer comprising the drug.

18. The transdermal patch as recited in claim 13, further comprising a release liner layer in contact with the adhesive layer and adapted to protect the adhesive layer prior to use of the transdermal patch.

19. The transdermal patch as recited in claim 13, wherein the layer comprising the drug comprises a drug reservoir layer.

20. The transdermal patch as recited in claim 19, further comprising a backing layer adapted to prevent escape of the drug from the transdermal patch other than into the skin, wherein the backing layer is positioned between the pigment layer and the drug reservoir layer.
